(19) United States
(12) Patent Application Publication
(10) Pub. No.: US 2018/0309230 A1
(43) Pub. Date: Oct. 25, 2018

(54) SELF-SEALING DEVICE AND METHODS OF USE

(71) Applicant: OHIO STATE INNOVATION FOUNDATION, Columbus, OH (US)

(72) Inventors: Ali REZAI, Morgantown, WV (US); Milind DEOGAONKAR, Westerville, OH (US)

(21) Appl. No.: 15/779,320

(22) PCT Filed: Nov. 25, 2016
(86) PCT No.: PCT/US2016/063758

§ 371 Date: May 25, 2018

Related U.S. Application Data
(60) Provisional application No. 62/259,984, filed on Nov. 25, 2015.

(51) Int. Cl. H01R 13/52; A61N 1/375; H01R 43/20

(52) U.S. Cl. CPC H01R 13/5216; H01R 43/20; H01R 13/5221; A61N 1/3752

(57) ABSTRACT

A self-sealing device includes an elastic sheet that is sized and dimensioned to cover a connector that joins a lead and a lead extension. The sheet has a first memory corresponding to an unfurled configuration and a second memory corresponding to a furled configuration. The sheet, when placed about the connector in the unfurled configuration, automatically obtains the furled configuration and covers the connector so that a fluid-tight seal is formed therebetween.

### RELATED APPLICATION

This application claims the benefit of U.S. Provisional Patent Application No. 62/259,984, filed Nov. 25, 2015, the entirety of which is hereby incorporated by reference for all purposes.

### TECHNICAL FIELD

The present disclosure relates generally to a device for lead implantation and, more particularly, to a self-sealing device and related methods for sealing a connector between a lead and a lead connector.

### BACKGROUND

Lead sealing mechanisms are commonly used to cover and seal a lead at a connector site. Such devices, such as the Stim-Loc™ connector cap device (available from Medtronic, Inc., Minneapolis, Minn.) can include a boot or a cap that moves retrograde over the lead before the connector is placed, and then transitions back over the connector after the connector is placed. However, during an implantation procedure of the lead and the connector, the lead must typically be moved relative to the cap so that the cap properly seals the connector site. During the implantation procedure, a larger incision can be required so that a portion of the lead is exposed, thereby increasing the chances of lead damage. Such implantation procedures can also be time-consuming and increase the risk of harm to a subject.

### SUMMARY

The present disclosure relates generally to a device for lead implantation and, more particularly, to a self-sealing device and related methods for sealing a connector between a lead and a lead connector.

One aspect of the present disclosure can relate to a self-sealing device that includes an elastic sheet that can be sized and dimensioned to cover a connector that joins a lead and a lead extension. The sheet can have a first memory corresponding to an unfurled configuration and a second memory corresponding to a furled configuration. The sheet, when placed about the connector in the unfurled configuration, can automatically obtain the furled configuration and covers the connector so that a fluid-tight seal is formed therebetween.

Another aspect of the present disclosure can include a system. The system can comprise a lead, a lead extension connected to the lead, a connector that operatively joins the lead to the lead extension, and a self-sealing device that covers the connector and forms a fluid-tight seal therebetween.

Another aspect of the present disclosure can relate to a method for sealing a connector that joins a lead and a lead extension. One step of the method can include providing an elastic sheet that is sized and dimensioned to cover the connector. The sheet can have a first memory corresponding to an unfurled configuration and a second memory corresponding to a furled configuration. The sheet, in the unfurled configuration, can be positioned about the connector. The sheet can be actuated so that the sheet obtains the furled configuration. The connector can be covered when the sheet obtains the furled configuration so that a fluid-tight seal is formed therebetween.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1A is a schematic illustration of a self-sealing device constructed in accordance with one aspect of the present disclosure;

FIG. 1B is a schematic illustration of a self-sealing device constructed in accordance with another aspect of the present disclosure;

FIG. 1C is a schematic illustration of a self-sealing device constructed in accordance with another aspect of the present disclosure;

FIG. 2 is a process flow diagram illustrating a method for sealing a connector that joins a lead and a lead extension according to another aspect of the present disclosure;

FIG. 3 is a schematic illustration showing the self-sealing device in FIG. 1 in an unfurled configuration positioned about a connector that joins a lead and a lead extension;

FIG. 4 is a schematic illustration showing the self-sealing device in FIG. 3 being actuated to transition from the unfurled configuration to a furled configuration; and

FIG. 5 is a schematic illustration showing the self-sealing device in FIG. 4 in the furled configuration wrapping around the connector.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the device in use or operation in addition to the orientation depicted in the figures. For example, if the device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “subject” can be used interchangeably with the term “patient” and refer to any warm-blooded organism including, but not limited to, human beings, pigs, rats, mice, dogs, goats, sheep, horses, monkeys, apes, farm animals, livestock, rabbits, cattle, etc.

As used herein, the term “operatively connected” can refer to an arrangement of the recited elements or components that establishes either a static connection between or a kinetic interaction between the recited elements or components, either by direct attachment of the elements or components together or by connection of the recited elements or components through one or more intervening elements or components. In one example, elements or components that are operatively connected to one another can mean that the elements or components are in electrical communication with one another.

As used herein, the term “electrical communication” can refer to the ability of a generated electric field to be transferred to, or have an effect on, one or more components of the present disclosure. In some instances, the generated electric field can be directly transferred to a component (e.g., via a wire or lead). In other instances, the generated electric field can be wirelessly transferred to a component.

### Overview

The present disclosure relates generally to a device for lead implantation and, more particularly, to a self-sealing device and related methods for sealing a connector between a lead and a lead connector. Conventional sealing devices can require movement of a lead to properly position the sealing device about a connector between the lead and a lead extension. An implantation procedure of a lead using such sealing devices requires a larger incision to expose the lead, thereby increasing: (1) the chances of lead damage; (2) the time of the implantation procedure; and (3) the risk of patient harm. Advantageously, the present disclosure provides a self-sealing device placed at a connector site of a lead and a lead extension that can: (1) be easy to place; (2) reduce exposure and risk of damage of the lead; (3) eliminate the need for an attachment mechanism (e.g., ties) that entails placement or securing via tactile means; and (4) completely seal around the connector without damaging the lead.

### Devices and Systems

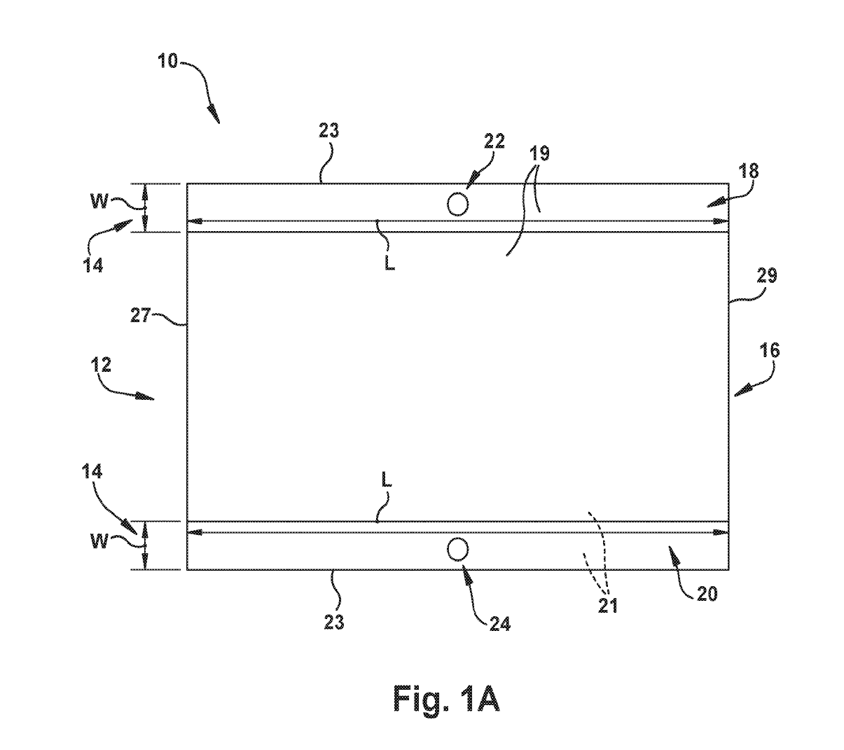

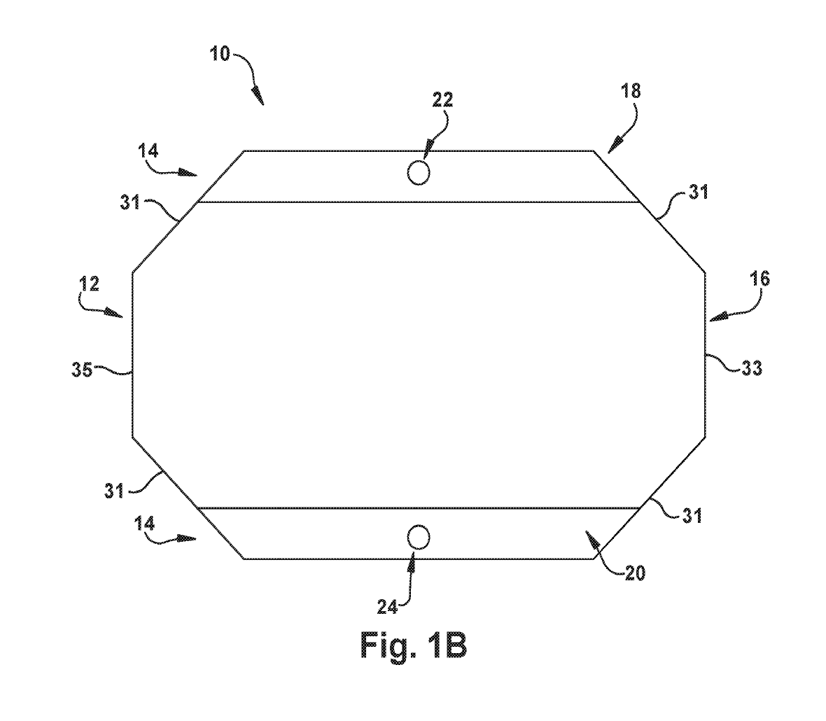

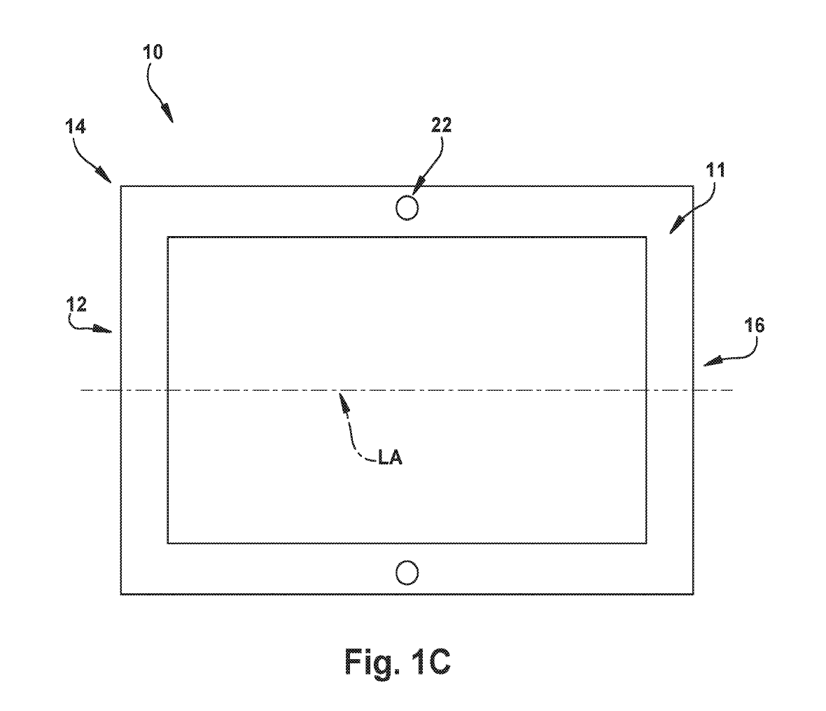

One aspect of the present disclosure can include a self-sealing device 10 (FIG. 1A). The self-sealing device 10 can comprise an elastic sheet 12 and a clamping mechanism 14. The sheet 12 can comprise a main body 16 having a first end portion 18 and an opposing second end portion 20. Each of the first and second end portions 18 and 20 comprises a sub-section of the sheet having a total surface area that is less than the total surface area of the entire sheet 12. As shown in FIG. 1A, for example, each of the first and second end portions 18 and 20 has a relatively thin, rectangular shape. Each of the first and second end portions 18 and 20 is defined by a length L and a width W. The length L of each of the first and second end portions 18 and 20 can be equal to, or about equal to, the length of main body portion 12 of the sheet 12, while the width W of each of the first and second end portions 18 and 20 is less than the width of the entire sheet.

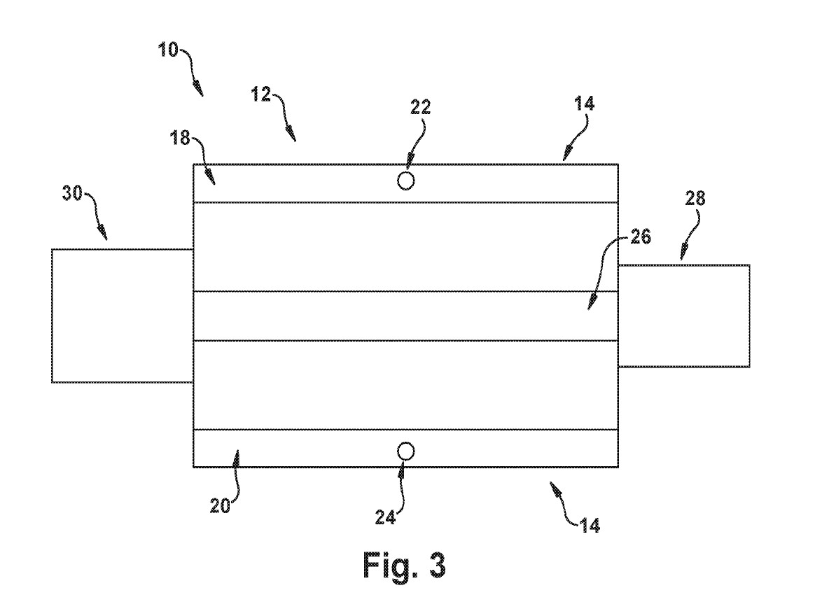

The sheet 12 can be defined by a first surface 19 and an oppositely disposed second surface 21. The sheet 12 can be made from one or more layers of a biocompatible, liquid-impermeable material, such as silicon (or a similar insulative material). In some instances, the sheet 12 can also include one or more layers of a self-recoil memory material (e.g., Silastic™). The sheet 12 can be sized and dimensioned to cover (e.g., entirely or partly) a connector 26 that joins a lead 28 and a lead extension 30 (FIG. 3). The sheet 12 can have a rectangular shape (or any other suitable shape) for sealing the connector 26. Where the sheet 12 has a rectangular shape, the sheet can be defined by first and second edges 23 and 25 that have an equal, or approximately equal, length that is greater than the length of third and fourth edges 27 and 29. The sheet 12 can have a flexible configuration for wrapping around the connector 26.

An alternative shape of the sheet 12 is shown in FIG. 1B. In this shape, which may be characterized as octagonal or elongated octagon, the sheet 12 can have a generally rectangular shape, except that the sheet includes four tapered edges 31. The tapered edges 31 are advantageous as they reduce the amount of material needed to construct the sheet 12 (relative to the rectangular shape) and thereby create a low profile construction about the ends 33 and 35 of the sheet 12 when the sheet is in the furled configuration.

The clamping mechanism 14 (FIG. 1A) can be defined by one or both of the first and second end portions 18 and 20. The clamping mechanism 14 functions to sealingly connect the first end portion 18 with the second end portion 20 when the first and second end portions are in direct contact with one another. In some instances, the first end portion 18 can overlie the second end portion 20 (or vice-versa) when the sheet 12 obtains the furled configuration. For example, one of the first and second end portions 18 and 20 can be physically connected to (e.g., in direct contact with) the other of the first and second end portions via the clamping mechanism 14.

The first end portion 18 can include one or more first clamping mechanisms 22 that cover, extend through, or are otherwise connected to, at least a portion thereof. The second end portion 20 can include one or more second clamping mechanisms 24 that cover, extend through, or are otherwise connected to, at least a portion thereof. In some instances, the first and second clamping mechanisms 22 and 24 can be configured to engage one another. For example, the first and second clamping mechanisms 22 and 24 can each comprise a cooperating element (e.g., a clamp, a snap, a hook and loop, an area of the sheet 12 that includes an opening extending between the surfaces 19 and 21, an adhesive layer, etc.) for engagement with each other.

In an alternative construction shown in FIG. 1C, the first and second end portions 18 and 20 comprise a single contact section 11 located about the perimeter of the sheet 12. In this configuration, the contact section 11 can contact a mirror image section of itself when folded over a longitudinal axis LA thereof to form a fluid-tight seal when the sheet 12 is in the furled configuration.

The sheet 12 can have a first memory of an unfurled configuration (e.g., a planar shape) and a second memory corresponding to a furled configuration (e.g., a non-planar shape, such as a circular shape when viewed in cross-section). The first and second end portions 18 and 20 can be spaced apart (e.g., radially spaced apart) from each other when the sheet 12 is in the unfurled configuration. The first and second end portions 18 and 20 can engage or directly contact each other when the sheet 12 is in the furled configuration. In some instances, one of the first or second end portions 18 or 20 can overlie the other of the first or second end portions when the sheet 12 is in the furled configuration.

The clamping mechanism 14 can be actuated in a number of different ways. In one example, the first and second end portions 18 and 20 can be automatically brought into direct contact with one another (to form the furled configuration) by virtue of the shape memory material used to construct the device 10. In other instances, the sheet 12 can be configured to obtain the furled configuration in response to a stimulus (e.g., application of heat, such as body heat). Advantageously, the sheet 12, when placed about the connector 26 (FIG. 3) in the unfurled configuration, automatically obtains the furled configuration and covers the connector so that a fluid-tight seal is formed therebetween without damaging the lead, thereby reducing exposure and risk of damage of the lead. This is also advantageous because it removes the need to form the device 10 around the connector by tactile means, which is very difficult given the small size of leads and lead connectors, as well as the precarious anatomy (e.g., neurological tissue) surrounding areas of lead placement and fixation.

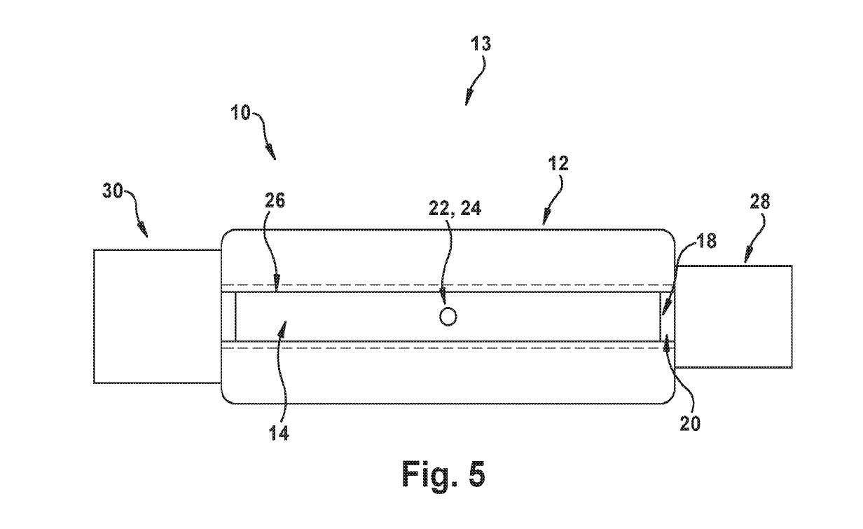

Another aspect of the present disclosure can include a system 13 (FIG. 5). The system 13 can comprise a lead 28, a lead extension 30 operatively connected to the lead, a connector 26 that operatively joins the lead to the lead extension, and a self-sealing device 10 that covers the connector and forms a fluid-tight seal therebetween.

### Methods

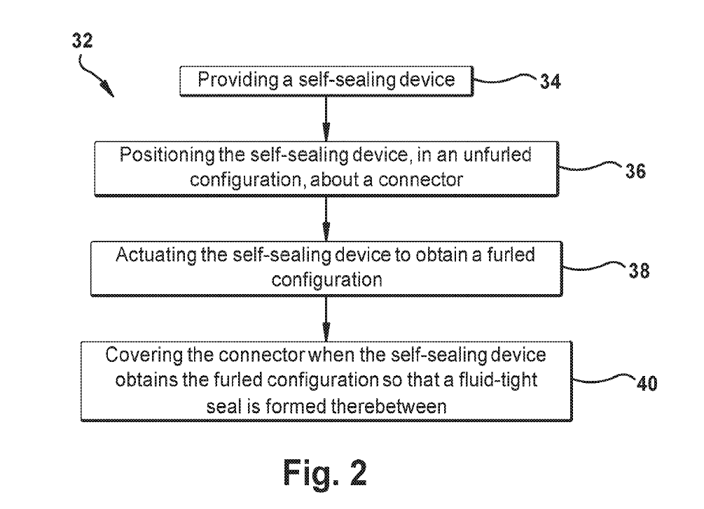

Another aspect of the present disclosure can include a method 32 (FIG. 2) for sealing a connector that joins a lead and a lead extension. The method 32 can generally include the steps of: providing a self-sealing device (Step 34); positioning the self-sealing device, in an unfurled configuration, about a connector (Step 36); actuating the self-sealing device to obtain a furled configuration (Step 38); and covering the connector when the self-sealing device obtains the furled configuration so that a fluid-tight seal is formed therebetween (Step 40). The method 32 can be used in a variety of lead implantation procedures (e.g., DBS lead implantations, spinal cord stimulator lead implantations, etc.).

At Step 34, a self-sealing device 10 can be provided. In some instances, the self-sealing device 10 can be configured as shown in FIG. 1 and described above. For example, the self-sealing device 10 can comprise an elastic sheet 12 having a first memory corresponding to an unfurled configuration and a second memory corresponding to a furled configuration. It will be appreciated that the particular dimensions of the self-sealing device 10 can depend upon the particular medical or surgical indication.

At Step 36, the sheet 12 of the self-sealing device 10 can be positioned, in the unfurled configuration, about a connector 26 (FIG. 3). The sheet 12 can be positioned under the connector 26 (or any suitable location relative to the connector). As shown in FIG. 2, the sheet 12 can be sized and dimensioned to underlie only the connector 26. It will be appreciated that the sheet 12 can be sized and dimensioned to underlie the connector 26 and a portion of each of the lead 28 and the lead extension 30.

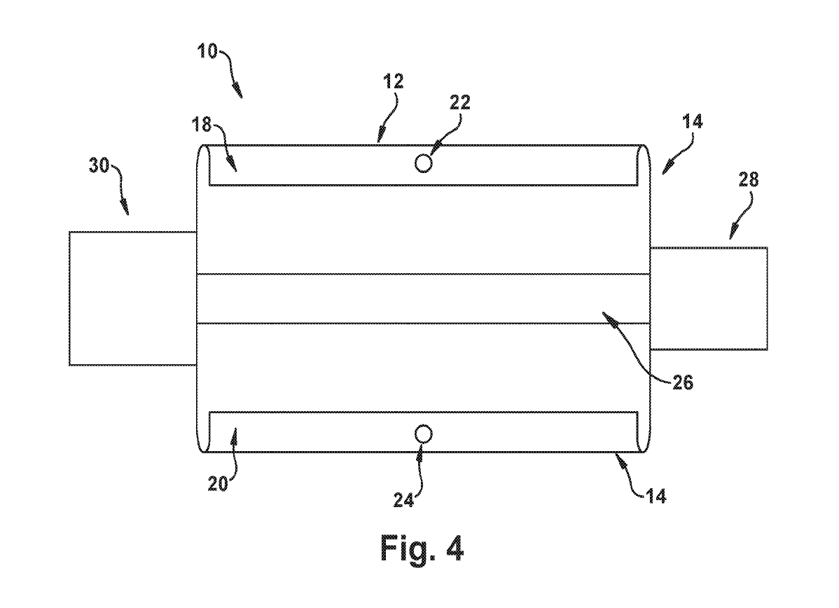

At Step 38, the sheet 12 can be actuated to obtain the furled configuration. In some instances, a stimulus can be applied to the sheet 12. In one example, the stimulus can be a tactile force produced by contacting the sheet 12 (e.g., with the connector 26, a medical instrument, a user's finger, etc.). In another example, the sheet 12 can include an actuating mechanism (not shown) operably connected to a portion of the sheet 12 that can be actuated to cause the sheet 12 to obtain the furled configuration. When the stimulus is applied to the sheet 12, the first and second end portions 18 and 20 can move towards each other to wrap around the connector 26 (FIG. 4). The sheet 12 can form a cylinder around the connector 26 to seal the connector. In one example, the first end portion 18 can overlie the second end portion 20 when the sheet 12 obtains the furled configuration. In another example, the second end portion 20 can overlie the first end portion 18 when the sheet 12 obtains the furled configuration. Advantageously, the wrap-around ability of the sheet 12 eliminates the need for an attachment mechanism (e.g., ties) to attach the sheet about the connector 26.

At Step 40, the sheet 12 can cover the connector 26 when the sheet obtains the furled configuration so that a fluid-tight seal is formed therebetween. In some instances, the first and second clamping mechanisms 22 and 24 can overlie one another when the sheet 12 is in the furled configuration (FIG. 5). In one example, the first and second clamping mechanisms 22 and 24 can be engaged with each other to help form the fluid-tight seal between the sheet 12 and the connector 26. In another example, a fastener (not shown) can be inserted through the overlying first and second clamping members 22 and 24.

From the above description of the present disclosure, those skilled in the art will perceive improvements, changes, and modifications. Such improvements, changes, and/or modifications are within the skill of the art and are intended to be covered by the appended claims.

1. A self-sealing device comprising:
an elastic sheet sized and dimensioned to cover a connector that joins a lead and a lead extension;
wherein the sheet has a first memory corresponding to an unfurled configuration and a second memory corresponding to a furled configuration;
whereby the sheet, when placed about the connector in the unfurled configuration, automatically obtains the furled configuration and covers the connector so that a fluid-tight seal is formed therebetween.

2. The self-sealing device of claim 1, wherein the sheet further comprises:
a main body having a first end portion and an opposing second end portion; and
a clamping mechanism associated with at least one of the first and second end portions;
wherein one of the first and second end portions overlies, and is physically connected with, the other of the first and second end portions via the clamping mechanism when the sheet obtains the furled configuration.

3. The self-sealing device of claim 2, wherein the clamping mechanism comprises a first clamping member operably connected to the first end portion of the main body and a second clamping member operably connected to the second end portion.

4. The self-sealing device of claim 3, wherein one of the first and second clamping members physically connects the other of the first and second clamping members when the sheet obtains the furled configuration.

5. The self-sealing device of claim 1, further configured to obtain the furled configuration in response to a stimulus.

6. A system comprising:
a lead;
a lead extension connected to the lead;
a connector that operatively joins the lead to the lead extension; and
a self-sealing device that covers the connector and forms a fluid-tight seal therebetween.

7. The system of claim 6, wherein the self-sealing device comprises:
an elastic sheet sized and dimensioned to cover the connector;
wherein the sheet has a first memory corresponding to an unfurled configuration and a second memory corresponding to a furled configuration;
whereby the sheet, when placed about the connector in the unfurled configuration, automatically obtains the furled configuration and covers the connector so that a fluid-tight seal is formed therebetween.

8. The system of claim 7, wherein the sheet further comprises:
a main body having a first end portion and an opposing second end portion; and
a clamping mechanism associated with at least one of the first and second end portions;
wherein one of the first and second end portions overlies, and is physically connected with, the other of the first and second end portions via the clamping mechanism when the sheet obtains the furled configuration.

9. The system of claim 8, wherein the clamping mechanism comprises a first clamping member operably connected to the first end portion of the main body and a second clamping member operably connected to the second end portion.

10. The system of claim 9, wherein one of the first and second clamping members physically connects the other of the first and second clamping members when the sheet obtains the furled configuration.

11. The system of claim 7, wherein the sheet is configured to obtain the furled configuration in response to a stimulus.

12. A method for sealing a connector that joins a lead and a lead extension, the method comprising the steps of:
providing a self-sealing device, the self-sealing device having an elastic sheet sized and dimensioned to cover the connector, the sheet having a first memory corresponding to an unfurled configuration and a second memory corresponding to a furled configuration;
positioning the sheet, in the unfurled configuration, about the connector;
actuating the sheet so that the sheet obtains the furled configuration; and
covering the connector when the sheet obtains the furled configuration so that a fluid-tight seal is formed therebetween.

13. The method of claim 12, wherein the step of providing a self-sealing device further comprises:
including a first clamping member operably connected to a first end portion of the sheet and a second clamping member operably connected to a second end portion of the sheet.

14. The method of claim 13, further comprising actuating the sheet to cause one of the first and second clamping members to overlie the other of the first and second clamping members when the sheet obtains the furled configuration.

15. The method of claim 12, wherein the step of actuating the sheet further comprises the step of:
applying a stimulus to the sheet.

16. A self-sealing device consisting of:
an elastic sheet sized and dimensioned to cover a connector that joins a lead and a lead extension;
wherein the sheet has a first memory corresponding to an unfurled configuration and a second memory corresponding to a furled configuration;
whereby the sheet, when placed about the connector in the unfurled configuration, automatically obtains the furled configuration and covers the connector so that a fluid-tight seal is formed therebetween.

17. The self-sealing device of claim 16, wherein the sheet further comprises:
a main body having a first end portion and an opposing second end portion; and
a clamping mechanism associated with at least one of the first and second end portions;
wherein one of the first and second end portions overlies, and is physically connected with, the other of the first and second end portions via the clamping mechanism when the sheet obtains the furled configuration.

18. The self-sealing device of claim 17, wherein the clamping mechanism comprises a first clamping member operably connected to the first end portion of the main body and a second clamping member operably connected to the second end portion.

19. The self-sealing device of claim 18, wherein one of the first and second clamping members physically connects the other of the first and second clamping members when the sheet obtains the furled configuration.

20. The self-sealing device of claim 16, further configured to obtain the furled configuration in response to a stimulus.
